(19) United States
(12) Patent Application Publication
(10) Pub. No.: US 2005/0176738 A1
(43) Pub. Date: Aug. 11, 2005

(54) Melanin-concentrating hormone receptor antagonists and compositions and methods related thereto

(75) Inventors: Val Goodfellow, Encinitas, CA (US); Mingzhu Zhang, San Diego, CA (US); Junko Tamiya, Oceanside, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 10/982,696

(22) Filed: Nov. 5, 2004

Related U.S. Application Data
(60) Provisional application No. 60/518,480, filed on Nov. 7, 2003.

(51) Int. Cl. A61K 31/522; A61K 31/519; C07D 498/02; C07D 473/02; C07D 487/02

(52) U.S. Cl. 514/260.1; 514/263.2; 514/264.1; 544/276; 544/278; 544/279

(57) ABSTRACT

Melanin-concentrating hormone (MCH) receptor antagonists are disclosed having utility for the treatment of MCH receptor-based disorders such as obesity. The compounds of this invention have the following structure: 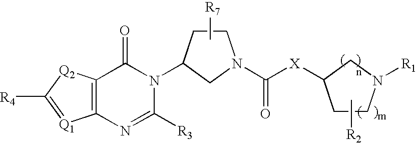
including pharmaceutically acceptable salts, esters, solvates, stereoisomers, and prodrugs thereof, wherein m, n, Q₁, Q₂, R₁, R₂, R₃, R₄, R₇ and X are as defined herein. Also disclosed are compositions containing a compound of this invention, as well as methods relating to the use thereof.

### CROSS-REFERENCE TO RELATED APPLICATION

This application claims priority to U.S. Provisional Application No. 60/518,480, filed Nov. 7, 2003, the entire disclosure of which is incorporated by reference herein.

### BACKGROUND OF THE INVENTION

1. Field of the Invention

This invention generally relates to antagonists of melanin-concentrating hormone receptors, and to compositions and methods related thereto.

2. Description of the Related Art

Melanin-concentrating hormone (MCH) is a neuropeptide that exerts a powerful effect on food intake and body weight regulation (Broberger & Hokfelt, PHYSIOL. BEHAV. 2001 November-December; 74(4-5): 669-82.) As a result, this neuropeptide, as well as antagonists to its various receptors, has been investigated for use in therapies relating to eating and body weight regulating disorders.

More specifically, MCH is a cyclic neuropeptide that is over-expressed in obese mice. Experiments where MCH was directly injected into lateral ventricles of the brains of rats resulted in increased consumption of food, indicating that MCH has a role in the regulation of body weight (Qu, et al., NATURE 1996 Mar. 21; 380 (6571): 243-7.) The orexigenic (appetite-stimulating) activity is believed to result from MCH's binding to a melanin-concentrating hormone receptor (MCH-1R) determined to be a 353 amino acid human orphan G-Protein-Coupled Receptor (GPCR) SLC-1 (Chambers et al., NATURE 1999 Jul. 15; 400(6741): 261-5; Saito et al., NATURE 1999 Jul. 15; 4000(6741): 265-9.) Mice deficient in MCH-1R have normal body weights, yet are lean and have reduced fat mass; thus, less susceptible to diet-induced obesity (Marsh et al., PROC. NATL. ACAD. SCI. 2002 Mar. 5; 99 (5): 3240-5.) A second MCH receptor (MCH-2R) has also been identified (Sailer et al., PROC. NATL. ACAD. SCI. 2001 Jun. 19; 98(13): 7564-9; An et al. PROC. NATL. ACAD. SCI. 2001 Jun. 19; 98(13): 7576-81.)

In view of its biological importance, a number of researchers have reported peptides or small molecule antagonists to MCH receptors. For example, Merck Research Laboratories has reported protein agonists consisting of the cyclic core of human MCH that activates both MCH-1R and MCH-2R, and an agonist with selectivity for MCH-1R (Bednarek et al., J BIOL CHEM 2002 Apr. 19; 277(16): 13821-6.) Takeda Chemical Industries (Takeda) has disclosed the use of (−)-N-[6-(dimethylamino)-methyl]-5,6,7,8-tetrahydro-2-naphthalenyl]-4′-fluoro-[1,1′-biphenyl]-4-carboxamide and derivatives thereof as selective MCH-1R inhibitors (Kakekawa et al., EUR J PHAMOCOL 2002 Mar. 8; 438(3); 129-35; WO 01/21577.) Additional Takeda patent publications directed to MCH antagonists include JP 2001226269; WO 01/21169; WO 01/82925; and WO 01/87834. Synaptic Pharmaceutical Corporation has similarly disclosed MCH receptor antagonists (WO 02/06245), as has Neurogen Corporation (WO 02/04433; U.S. 20020052383 A1.)

Accordingly, there remains a need in the art for novel MCH receptor antagonists, including antagonists of MCH-1R and/or MCH-2R, and for compositions and methods related thereto. The present invention fulfils these needs and provides further related advantages.

### BRIEF SUMMARY OF THE INVENTION

In brief, this invention is directed generally to compounds that function as antagonists to one or more melanin-concentrating hormone (MCH) receptor(s), such as, for example, MCH-1R and MCH-2R. This invention is also directed to compositions containing one or more of such compounds in combination with one or more pharmaceutically acceptable carriers, as well as to methods for treating conditions or disorders associated with MCH.

In one embodiment, this invention is directed to compounds that have the following structure (I): 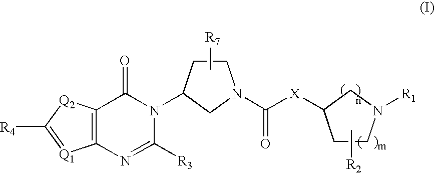
including pharmaceutically acceptable salts, esters, solvates, stereoisomers, and prodrugs thereof, wherein m, n, Q₁, Q₂, R₁, R₂, R₃, R₄, R₇ and X are as defined herein.

The compounds of this invention may have utility over a broad range of therapeutic applications, and may be used to treat disorders or illnesses, including (but not limited to) eating disorders, body weight disorders, anxiety, depression and CNS disorders. A representative method of treating such a disorder or illness includes administering an effective amount of a compound of this invention, typically in the form of a pharmaceutical composition, to an animal in need thereof (also referred to herein as a “patient,” including a human.)

Accordingly, in another embodiment, pharmaceutical compositions are disclosed containing one or more compounds of this invention in combination with a pharmaceutically acceptable carrier.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, the present invention is generally directed to compounds useful as melanin-concentrating hormone (MCH) receptor antagonists. The compounds of this invention have the following structure (I): 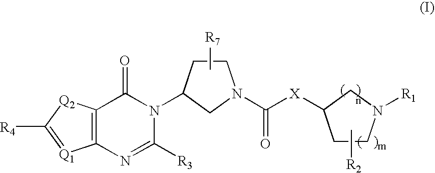
and pharmaceutically acceptable salts, esters, solvates, stereoisomers, and prodrugs thereof,
wherein:
   m is 0 or 1;
   n is 1 or 2;
   Q₁ is ═CH— or ═N—;
   Q₂ is —S—, —O—, —C(R₅)═C(R₆)—, —C(R₅)═N—, or —N═C(R₆)—;
   R₁ is alkyl, substituted alkyl, aryl, substituted aryl, heteroaryl, substituted heteroaryl, heteroarylalkyl, substituted heteroarylalkyl, arylalkyl, substituted arylalkyl, heterocyclealkyl, or substituted heterocyclealkyl;
   R₃ is hydrogen, halogen, cyano, alkyl, substituted alkyl, arylalkyl, or substituted arylalkyl;
   R₄ is hydrogen, alkyl, substituted alkyl, aryl, substituted aryl, arylalkyl, substituted arylalkyl, heterocycle, substituted heterocycle, heterocyclealkyl, or substituted heterocyclealkyl;
   R₂, R₅, R₆ and R₇ are the same or different and independently hydrogen, alkyl, or substituted alkyl;
   X is —CH₂— or —N(R₈)—; and
   R₈ is hydrogen, alkyl, or substituted alkyl.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂— cyclohexenyl, and the like. Cyclic alkyls are also referred to herein as a “homocycle” or “homocyclic ring.” Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂-phenyl), —(CH₂)₂-phenyl, —(CH₂)₃-phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle moiety, such as —CH₂-pyridinyl, —CH₂-pyrimidinyl, and the like.

The term “substituted” as used herein means any of the above groups (i.e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, sulfonylalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

“Cyano” means —CN.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Sulfonylalkyl” means an alkyl moiety attached through a sulfonyl bridge (i.e., —SO₂-alkyl) such as methylsulfonyl, ethylsulfonyl, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

“Hydroxyalkyl” means an alkyl substituted with at least one hydroxyl group.

In one embodiment, compounds of this invention have the following structure (II) when X is —CH₂— and structure (III) when X is —N(R₉)—:

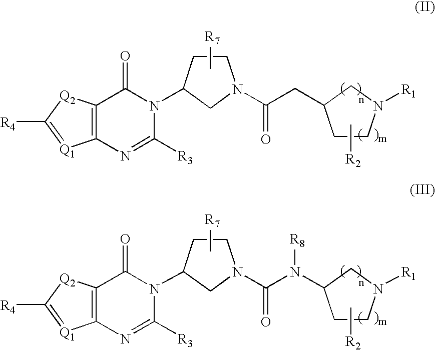

In other embodiments, compounds of this invention have the following structure (IV) when m is 0 and n is 1, structure (V) when m is 1 and n is 1 or when m is 0 and n is 2, and structure (VI) when m is 1 and n is 2:

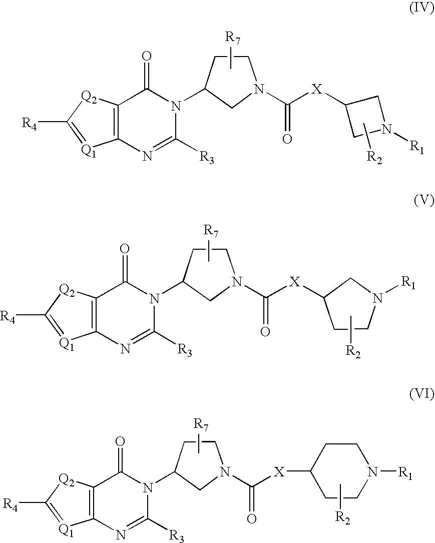

In further embodiments, compounds of this invention have the following structure (VII) when X is —CH₂— and both m and n are 1, and structure (VIII) when X is —N(R₈)— and both m and n are 1:

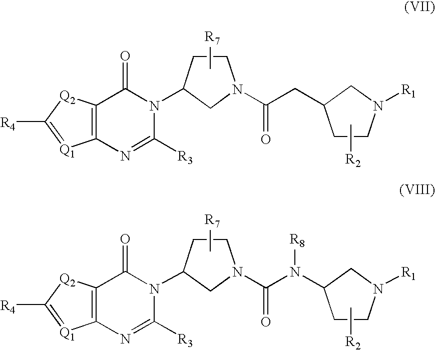

In further embodiments of structures (VII) and (VIII), R₂ and R₇ are hydrogen, and compounds of this invention have the following structures (IX) and (X), respectively:

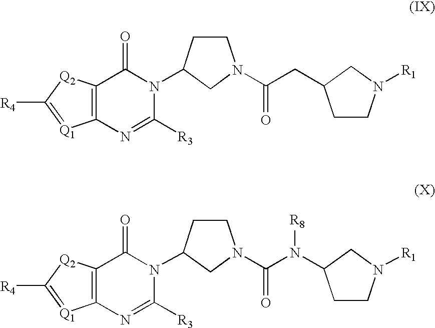

In further embodiments, compounds of this invention wherein Q₁ is ═CH—, Q₂ is —S—, R₂ and R₇ are hydrogen, and both m and n are 1, have the following structure (XI) when X is —CH₂— and structure (XII) when X is —N(R₈)—:

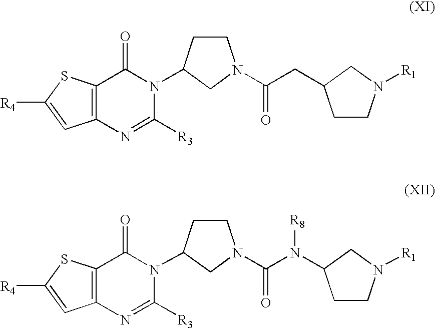

In further embodiments, compounds of this invention wherein Q₁ is N, Q₂ is S, R₂ and R₇ are hydrogen, and both m and n are 1, have the following structure (XIII) when X is —CH₂— and structure (XIV) when X is —N(R₈)—:

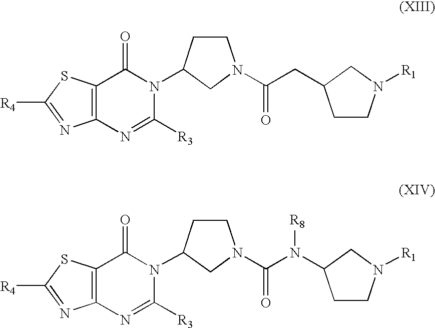

In further embodiments, compounds of this invention wherein Q₁ is —CH—, Q₂ is —O—, R₂ and R₇ are hydrogen, and both m and n are 1, have the following structure (XV) when X is —CH₂— and structure (XVI) when X is —N(R₈)—:

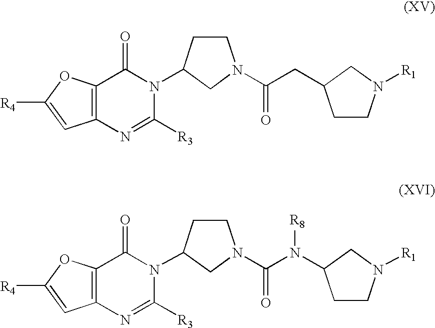

In further embodiments, compounds of this invention wherein Q₁ is ═CH—, Q₂ is —C(R₅)═C(R₆)—, R₂, R₅, R₆, and R₇ are hydrogen, and both m and n are 1, have the following structure (XVII) when X is —CH₂— and structure (XVIII) when X is —N(R₈)—:

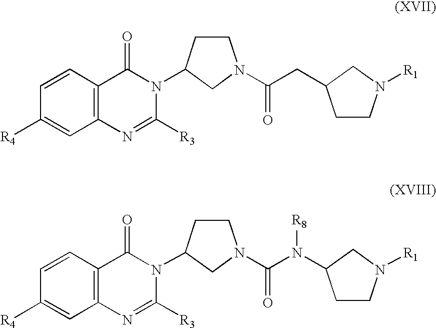

In further embodiments, compounds of this invention wherein Q₁ is ═N—, Q₂ is —C(R₅)═C(R₆)—, R₂, R₅, R₆ and R₇ are hydrogen, and both m and n are 1, have the following structure (XIX) when X is —CH₂— and structure (XX) when X is —N(R₈)—:

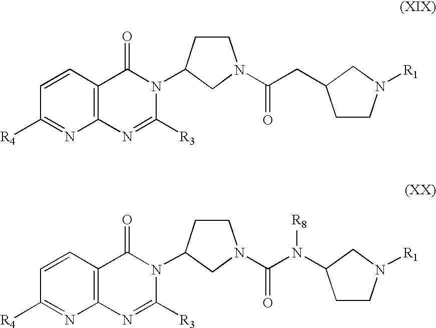

In further embodiments, compounds of this invention wherein Q₁ is —CH—, Q₂ is —N═C(R₆)—, R₂, R₆ and R₇ are hydrogen, and both m and n are 1, have the following structure (XXI) when X is —CH₂— and structure (XXII) when X is —N(R₈)—:

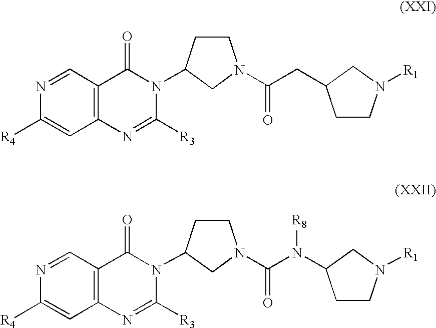

In further embodiments, compounds of this invention wherein Q₁ is N, Q₂ is —C(R₅)═N—, R₂ and R₇ are hydrogen, and both m and n are 1, have the following structure (XXIII) when X is —CH₂— and structure (XXIV) when X is —N(R₈)—:

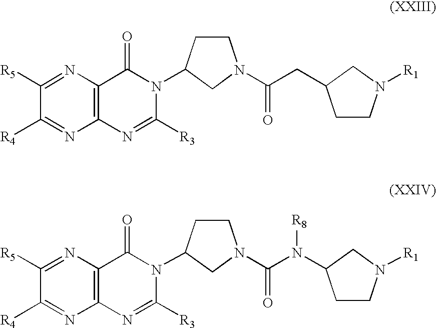

In further embodiments, compounds of this invention wherein Q₁ is ═N—, Q₂ is —(N═CR₆)—, R₂, R₆, and R₇ are hydrogen, and both m and n are 1, have the following structure (XXV) when X is —CH₂— and structure (XXVI) when X is —N(R₈)—:

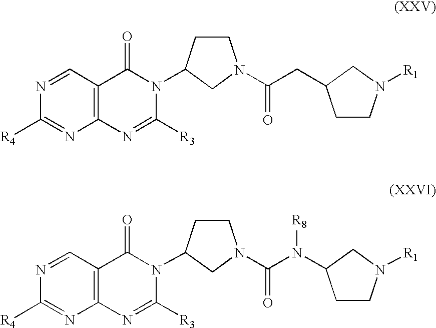

In further embodiments, R₁ and R₄ are alkyl or substituted alkyl, wherein the alkyl moiety, as well as the alkyl portion of substituted alkyl moiety, includes saturated straight chain and saturated branched alkyls, as well as saturated cyclic alkyls such as cyclohexyl. In this context, substituted alkyls include alkyls substituted with one or more substituents as defined above, including (but not limited to) —ORₐ, —SRₐ, —C(═O)Rₐ, —S(═O)Rₐ, —S(═O)₂Rₐ, and —S(═O)₂NRₐR_{b}, wherein Rₐ and R_{b} are as defined above and including (but not limited to) alkyl, aryl and heterocycle optionally substituted with a one or more further substituent(s) as defined above. For example, representative substituted R₁ moieties include alkyl substituted with —O(alkyl), —S(alkyl), —C(═O)(alkyl), —S(═O)(alkyl), —S(═O)₂(alkyl), —O(aryl), —S(aryl), —C(═O)(aryl), —S(═O)(aryl), —S(═O)₂(aryl), —O(heterocycle), —S(heterocycle), —C(═O)(heterocycle), —S(═O)(heterocycle), and —S(═O)₂(heterocycle), wherein each of alkyl, aryl and heterocycle may be further substituted with one or more substituents.

In other embodiments, R₁ is alkyl, substituted alkyl, arylalkyl, substituted arylalkyl, heterocyclealkyl, or substituted heterocyclealkyl; and R₄ is hydrogen, alkyl, substituted alkyl, aryl, substituted aryl, arylalkyl, substituted arylalkyl, heterocycle, or substituted heterocycle.

Representative R₂ and R₇ moieties include, but are not limited to, hydrogen.

Representative R₃ moieties include lower alkyl, including, but not limited to, lower straight chain alkyls such as methyl, ethyl and propyl.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I.) Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality that may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be prepared by known organic synthesis techniques, including the methods described in more detail in the Reaction Schemes and in the Examples.

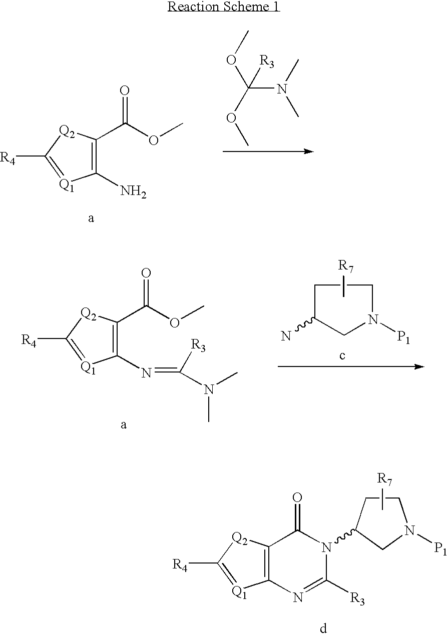

The 5-substituted 3-amino-2-carboxyl methyl ester pentacyclic ring a is refluxed with N,N-dimethylalkylamide dimethyl acetal yielding methyl ester b. To compound b is added substituted protected 3-aminopyrrolidine c of defined chirality. The protecting group P₁ includes (but is not limited to) Boc. This reaction yields the protected 3-pyrrolidin-3-yl-bicyclo-pyrimidin-4-one d of chirality specified by the chirality of compound c.

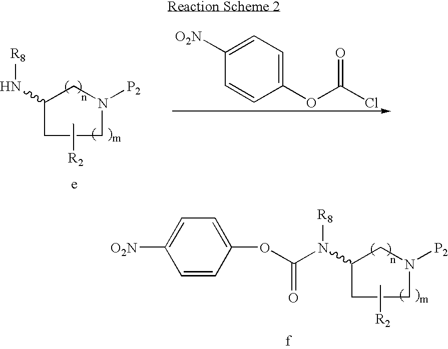

p-Nitrophenylchlorocarbonate reacts with protected amine pyrrolidine e to yield protected pyrrolidine carbamate ester f which serves as reagent for subsequent reactions. The protecting group P₂ can be (but is not limited to) Boc or benzyl.

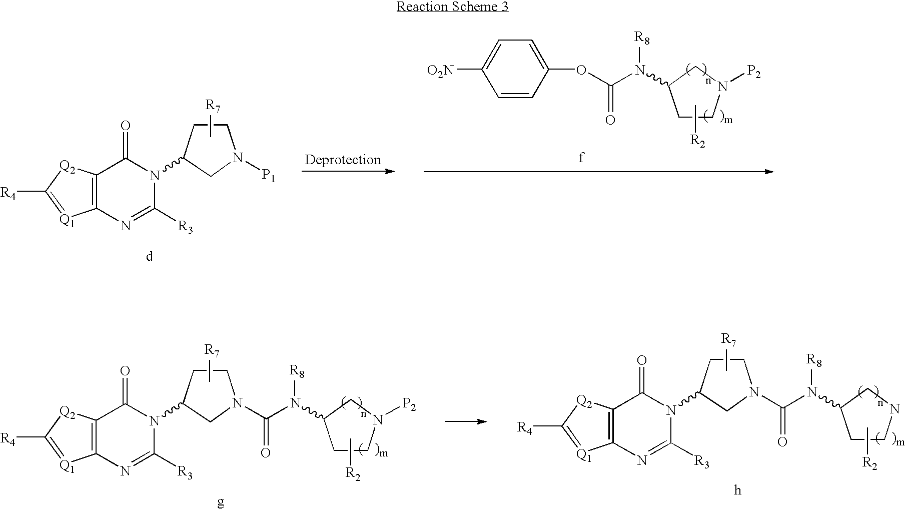

After removal of the protecting group P₁ of compound d, the resulting species reacts with protected pyrrolidine carbamate ester f (Reaction Scheme 2) to yield protected bis-pyrrolidine bicyclic g. Deprotection by removal of protecting group P₂ results in the bis-pyrrolidine bicyclic h.

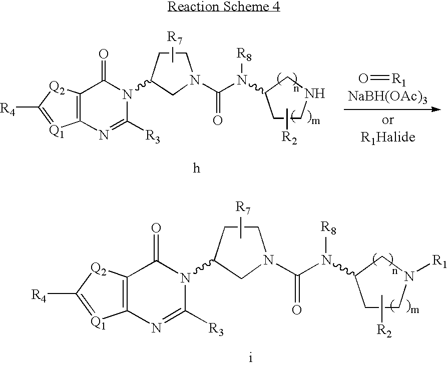

Bis-pyrrolidine bicyclic h is alkylated at the terminal pyrrolidine nitrogen via reductive amination or reaction with alkyl halide to yield compound i.

The compounds of this invention may be evaluated for their ability to bind to a MCH receptor by techniques known in this field. For example, a compound may be evaluated for MCH receptor binding by monitoring the displacement of an iodonated peptide ligand, typically human [¹²⁵,]-MCH, from cells expressing individual melanin concentrating hormone receptor subtypes. To this end, whole cells expressing the desired melanin concentrating hormone receptor are subjected to nitrogen cavitation, and the membrane fraction is isolated by differential centrifugation. Stock solutions of test compounds are diluted serially in binding buffer (50 mM HEPES+10 mM MgCl₂+2 mM EGTA) and an equal volume mixed with [¹²⁵I]-MCH (0.2 nM final) diluted in binding buffer. Unlabeled MCH is included as a control. Membranes (5-10 μg total protein) are added to each test compound concentration and incubated for 30 minutes at room temperature. Bound radioligand is captured using GF/C glass fiber filter plates treated with 1% PEI and coated with 1% BSA. Free radioligand is removed by three sequential washes with wash buffer (PBS+0.01% Triton X-100.) Kᵢ values are determined by data analysis using appropriate software, such as GraphPad Prizm, and data are plotted as counts of radiolabeled MCH bound versus the log concentration of test compound.

In addition, functional assays of receptor activation have been defined for the MCH receptors based on their coupling to Gq proteins. In response to MCH peptides, the MCH receptors couple to Gq and activate phospholipase C resulting in an increased release of intracellular calcium. Melanin concentrating hormone receptor activity can be measured in HEK293 cells expressing individual melanin concentrating hormone receptors by direct measurement of Ca²⁺ levels. For example, HEK293 cells expressing the desired MCH receptor are seeded into 96-well microtiter Poly-D-Lysine-coated plates at a density of 80,000 cells per well and allowed to adhere overnight with incubation at 37° C. in 5% CO₂. Test compounds are diluted in dilution buffer (HBSS+20 mM HEPES+0.1% BSA+2.5 mM Probenecid) and assessed for antagonist activity over a range of concentrations along with a control agonist MCH. Prior to the assay, cells are loaded with the calcium sensitive dye Fluo-4 for 1 hour at 37° C. Cells are then washed three times with assay buffer (dilution buffer without BSA), and brought to a final volume of 150 μl/well in assay buffer. At the time of assay, 50 μl of test compound is added to each well and allowed to incubate for 2 minutes at room temperature. MCH agonist peptide at a concentration of 10 nM is then added, and intracellular calcium release is measured in real-time using a fluorimetric imaging plate reader (FLIPR.) EC₅₀ values are determined by data analysis using appropriate software such as GraphPad Prizm, and data are plotted as relative fluorescent units produced versus log concentration of compound.

As mentioned above, the compounds of this invention may function as antagonists to the MCH receptor 1, and therefore may be useful in the treatment of a variety of conditions or diseases including (but not limited to) eating disorders and obesity. The compounds of the present invention may also be used in combination therapy with agents that modify food intake or appetite, and are also included within the scope of this invention. Such agents include, but are not limited to, other MCH receptor ligands, or ligands of the leptin, NPY, melanocortin, serotonin or B₃ adrenergic receptors.

In another embodiment, compounds of this invention may be useful as anti-anxiety and/or anti-depression agents through interaction with the MCH receptor. These compounds may also be used in combination therapy with other anti-anxiety agents or anti-psychotics for the treatment of anxiety, depression, schizophrenia, and other CNS diseases.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount that is effective to treat a particular disorder of interest. Typically, the pharmaceutical composition may include a compound of this invention in an amount ranging from 0.1 mg to 250 mg per dosage depending upon the route of administration, and more typically from 1 mg to 60 mg. One skilled in the art can readily determine appropriate concentrations and dosages.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface-active agents, binders, and lubricants. One skilled in this art may further formulate the compound in an appropriate manner, and in accordance with accepted practices, such as those disclosed in REMINGTON'S PHARMACEUTICAL SCIENCES, Gennaro, Ed., Mack Publishing Co., Easton, Pa. 1990.

In another embodiment, the present invention provides a method for treating a condition related to an MC receptor. Such conditions may include, for example, obesity, anxiety and depression. The methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Analytical Method 1: HPLC-MS

Platform: HP 1100 series: equipped with an auto-sampler, an UV detector (220 nM and 254 nM), a MS detector (electrospray);
Column: YMC ODS AQ, S-5, 5 μ, 2.0×50 mm cartridge;
HPLC gradients: 1.5 mL/minute, from 10% acetonitrile in water to 90% acetonitrile in water in 2.5 minutes, maintaining 90% for 1 minute.
   Analytical Method 2: Supercritical Fluid Chromatography (SFC)
Platform: Berger FCM1200 SFC pump, Agilent Diode Array Detector, Agilent Model 220 Microplate autosampler, Agilent Model 1946 MSD (APCI interface);
Column: Berger Pyridine 60A, 4 micron, 3×150 mm;
Solvents: SFC Grade CO₂, Optima-grade methanol with 1.5% water and 0.025% ethanesulfonic acid;
Flow rate: 4.0 mL/min, 120 Bar backpressure;
Gradient: 5-55% methanol/CO₂ in 2.4 min.
   Prep. HPLC-MS
Gilson HPLC-MS equipped with Gilson 215 auto-sampler/fraction collector, an UV detector and a ThermoFinnigan AQA Single QUAD Mass detector (electrospray);
HPLC column: BHK ODS-O/B, 5 μ, 30×75 mm
HPLC gradients: 35 mL/minute, 10% acetonitrile in water to 100% acetonitrile in 7 minutes, maintaining 100% acetonitrile for 3 minutes.
   Abbreviations:
Boc-Phe-CHO: (S)-(tertbutoxycarbonylamino)-3-phenylpropional
BOC: tert-butoxycarbonyl
DCM: dichloromethane
DMF: dimethylformamide
DMFDMA: N,N-dimethylformamide dimethyl acetal
DMSO: dimethylsulfoxide
EDC: 1-(3-dimethylaminopropyl)-3-ethylcarbodiimide hydrochloride
FMOC: N-(9-fluorenylmethoxycarbonyl)
HOBt: 1-hydroxybenzotriazole hydrate
HBTU:O-(1H-Benzotriazol-1-yl)-N,N,N′,N′-tetramethyluroniumhexafluorophosphate
NaBH(OAc)₃: Sodium Triacetoxyborohydride
Pd-C: Palladium (10%) on Carbon
TFA: Trifluoroacetic acid
TFAA: Trifluoroacetic anhydride
LDA: Lithium diisopropylamide
IsoPrOH: Isopropanol

### EXAMPLE 1

### (S)-3-[6-(4-CHLORO-PHENYL)-4-OXO-4H-THIENO[3,2-D]PYRIMIDIN-3-YL]-PYRROLIDINE-1-CARBOXYLIC ACID [(R)-1-(4,4-DIMETHYL-CYCLOHEXYL)-PYRROLIDIN-3-YL]-METHYL-AMIDE

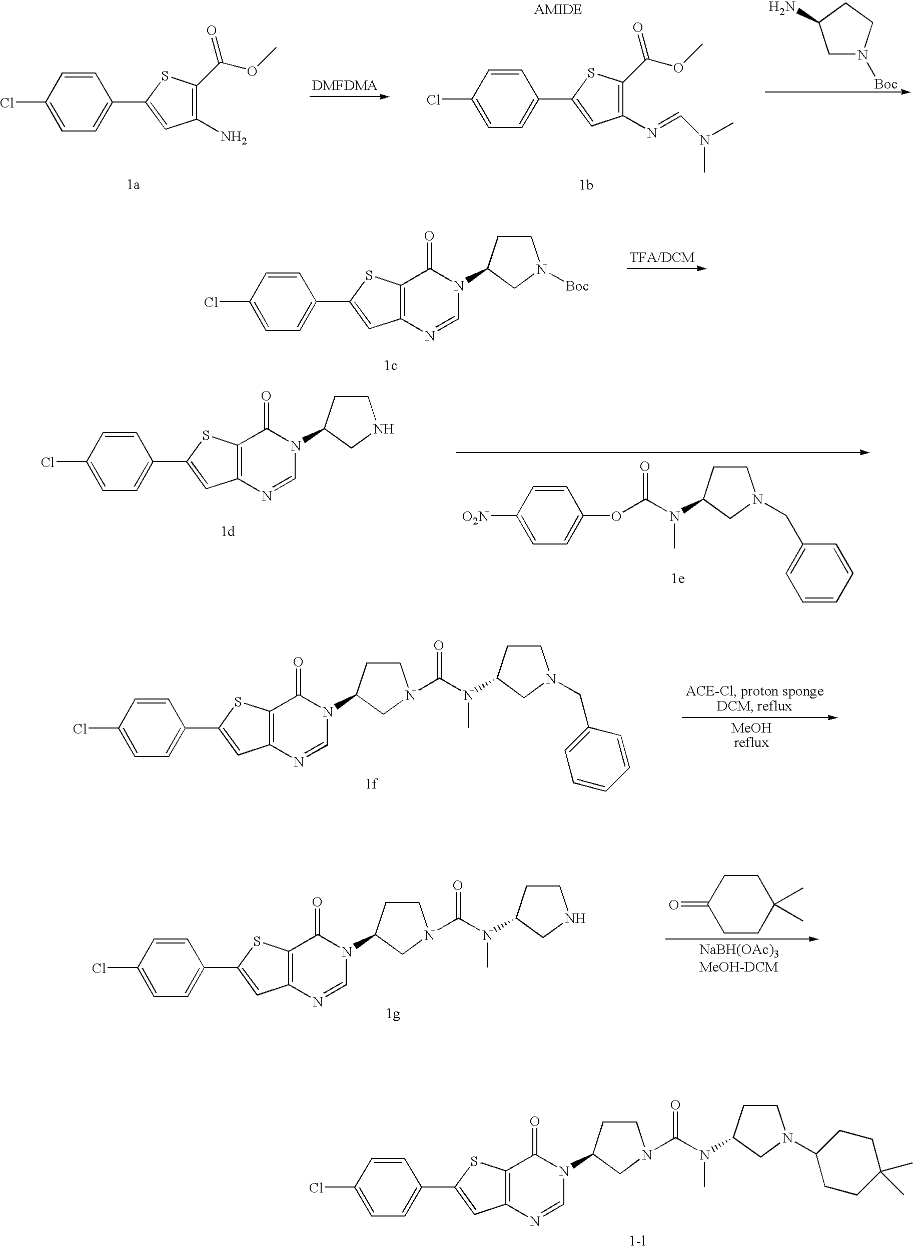 Step 1A:

To methyl-3-amino-5-(4-chlorophenyl)-2-thiophene carboxylate 1a (1.6 g, 6 mmol) in 20 mL ethanol was added dimethylformamide dimethyl acetal (2.0 mL, 15 mmol). The reaction mixture was refluxed in a 90° C. oil bath for 3 hours. Removal of solvent and excess dimethylformamide dimethyl acetal gave compound 1b as a brown oil.

Step 1B:

To compound 1b resulting from the previous step in 20 mL ethanol was added (3S)-(−)-Boc-3-aminopyrrolidine (1.2 g, 6.45 mmol). The reaction mixture was refluxed in a 100° C. oil bath overnight and then cooled to 4° C. overnight. The resulting solid was filtered and washed with cold ethanol to give 1.5 g of the Boc protected compound 1c. LC/MS: 432.0 [MH⁺].

Step 1C:

Compound 1c (1.5 g, 3.47 mmol) in 25 mL DCM was cooled in an ice bath. TFA was added with stirring for 1 hr to effect removal of the Boc protecting group. After removal of excess TFA by coevaporation with DCM, the resulting oil was dissolved in 100 mL DCM-isoPrOH and treated with 50 mL saturated NaHCO₃ solution. The organic layer was separated and the water layer was extracted with 50 mL DCM-isoPrOH. The combined organic layer was dried over MgSO₄ and evaporated to yield the unprotected 3-pyrrolidin-3-yl-3H-thieno[3,2-d]pyrimidin-4-one 1d (1.1 g).

Step 1D:

p-Nitrophenyl chlorocarbonate (5.7 g) was dissolved in 60 mL THF and chilled to 0° C. A solution of ((R)-1-benzyl-pyrrolidin-3-yl)methylamine (4.5 g) in 10 mL dry THF was added dropwise and the mixture was stirred at room temperature for 30 minutes. The reaction mix was filtered, washed with a small amount of THF, and dried to give the hydrochloride salt of ((R)-1-benzylpyrrolidin-3-yl)-methyl-carbamic acid 4-nitrophenyl ester 1e (8.9 g).

Compound 1e (1.38 g) and triethylamine (1.96 mL) were added to a solution of compound 1d (1.10 g) in DMF (10 mL). The reaction mixture was heated at 90° C. for 2 days and then cooled at 4° C. overnight. The resulted solid was filtered and washed with cold methanol to give compound 1f (0.68 g). LC/MS: 548.1 [MH⁺].

Step 1E:

Compound 1f (0.63 g) was dissolved in 30 mL DCM (anhydrous) together with proton-sponge (0.25 g). 1-Chloroethyl chloroformate (ACE-Cl, 0.25 g) was added and the mixture was stirred for 15 minutes and was refluxed for 1.5 hours. After removal of solvent, the residue was dissolved in 30 mL MeOH and refluxed for 1 hr. The reaction mixture was concentrated, precipitated with ether, filtered and washed with ether to give compound 1g (0.4 g). MS: [MH⁺]: 458.0.

Step 1F:

To compound 1g (30 mg) dissolved in 1 mL MeOH-DCM was added a catalytic amount of HOAc, two equivalents of NaBH(OAc)₃, and 2 equivalents of 4,4-dimethylcyclohexanone. The mixture was stirred for 1 hr at room temperature prior to quenching with 0.1 mL H₂O. The reaction mixture was filtered and purified by preparative LC/MS to give compound 1-1 (MS: [MH⁺]: 568.2.)

By varying the substitutions of the starting thiophene 1a and the alkylation reagent employed in Step 1F, the following compounds were prepared:

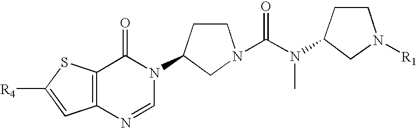

| Cpd. | R₄ | R₁ | MW | MS | RT* |
|---|---|---|---|---|---|
| 1-1 | 4-Cl—Ph— | 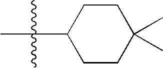 | 568.182 | 568.2 | 6.792 |
| 1-2 | 4-Cl—Ph— | 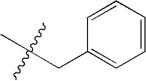 | 548.108 | 548.1 | 6.278 |
| 1-3 | 4-Cl—Ph— | H | 457.984 | 458.0 | 5.310 |
| 1-4 | 4-Cl—Ph— | 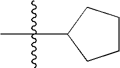 | 526.102 | 526.1 | 5.946 |
| 1-5 | 4-Cl—Ph— | 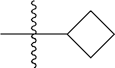 | 512.075 | 512.1 | 5.665 |
| 1-6 | 4-Cl—Ph— | 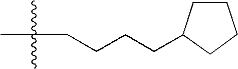 | 568.182 | 568.2 | 7.195 |
| 1-7 | 4-Cl—Ph— | 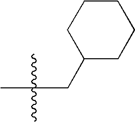 | 554.155 | 554.1 | 6.765 |
| 1-8 | 4-Cl—Ph— | 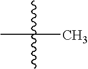 | 472.01 | 472.1 | 5.387 |
| 1-9 | 4-Cl—Ph— | 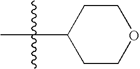 | 542.101 | 542.1 | 5.506 |
| 1-10 | 4-Cl—Ph— | 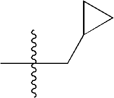 | 512.075 | 512.1 | 5.816 |
| 1-11 | 4-Cl—Ph— | 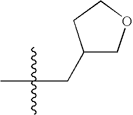 | 542.101 | 542.1 | 5.565 |
| 1-12 | 4-Cl—Ph— | 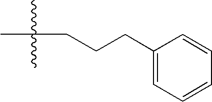 | 576.162 | 576.2 | 6.718 |
| 1-13 | 4-F—Ph— | 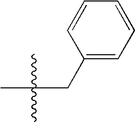 | 531.653 | 532.1 | 5.326 |

*All retention times (RT) reported for Analytical Method 1.

### EXAMPLE 2

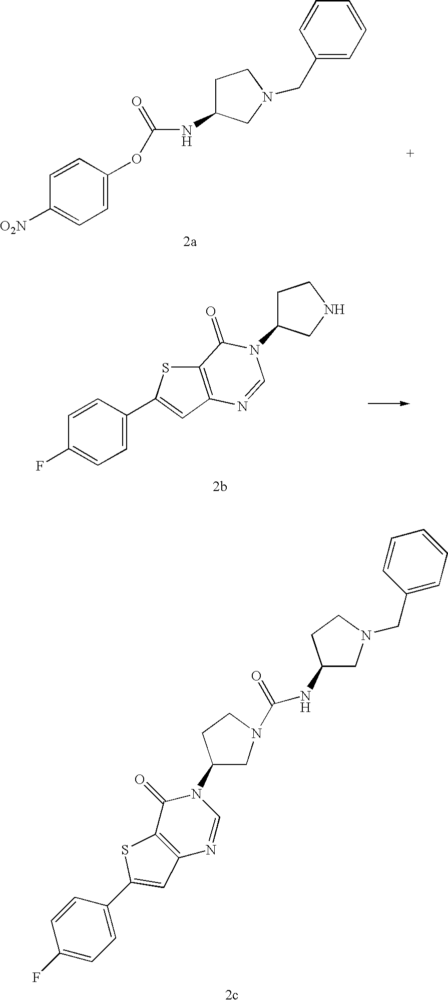 Step 2A:

The chirality of the invention can be determined by the use of chiral reagents. In the current example, the S-enantiomer of the benzyl-pyrrolidine carbamic acid compound 2a was synthesized starting from (S)-1-benzyl-pyrrolidin-3-yl-amine according to the method of Step 1D used in Example 1. Compound 2b was obtained starting with methyl-3-amino-5-(4-fluorophenyl)-2-thiophene carboxylate by the method of Steps 1A through 1C. Compounds 2a and 2b were coupled according to the procedure of Step 1D to give protected compound 2c. Using compound 2c and following the methods of Steps 1E for deprotection and 1F for alkylation, compounds of the following table were prepared:

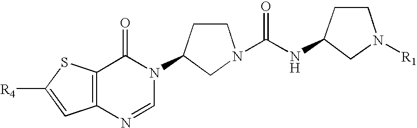

| Cpd. | R₄ | R₁ | MW | MS | RT* |
|---|---|---|---|---|---|
| 2-1 | 4-F—Ph— | 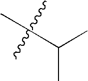 | 469.582 | 469.8 | 1.704 |
| 2-2 | 4-F—Ph— | 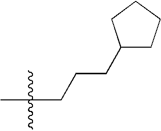 | 537.700 | 537.8 | 1.627 |
| 2-3 | 4-F—Ph— | 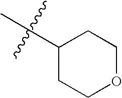 | 511.619 | 511.7 | 1.799 |
| 2-4 | 4-F—Ph— | 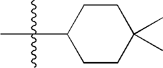 | 537.700 | 537.8 | 1.606 |

*All retention times (RT) reported for Analytical Method 2.

### EXAMPLE 3

### (S)-3-[6-(4-FLUORO-PHENYL)-2-METHYL-4-oxo-4H-THIENO[3,2-D]PYRIMIDIN-3-YL]-PYRROLIDINE-1-CARBOXYLIC ACID METHYL-[(S)-1-(3-PHENYL-PROPYL)-PYRROLIDIN-3-YL]-AMIDE

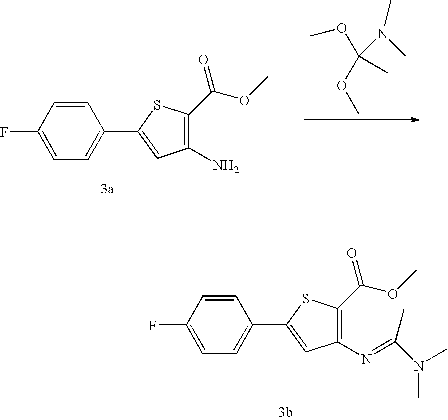 Step 3A:

Alkylation at the 2-position of the pyrimidine ring of the invention has been achieved by the incorporation of alkylated reagent prior to the ring closure reaction disclosed in Example 1 (Step 1B.) In the current example, thiophene 3a was refluxed with (1,1-dimethoxy-ethyl)-N,N-dimethyl-amine to afford after purification 5-(4-Chloro-phenyl)-3-[1-dimethylamino-eth-(E)-ylideneamino]-thiophene-2-carboxylic acid methyl ester, compound 3b. Compound 3b was then subjected to the reactions described in Example 1, Steps 1B-1F to afford compound 3-1.

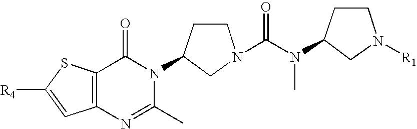

| Cpd. | R₄ | R₁ | MW | MS | RT* |
|---|---|---|---|---|---|
| 3-1 | 4-F—Ph— | 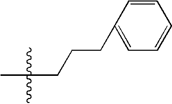 | 573.733 | 574.2 | 6.084 |

*All retention times (RT) reported for Analytical Method 1.

### EXAMPLE 4

### (S)-3-[6-(4-CHLORO-PHENYL)-4-OXO-4H-THIENO [3,2-D]PYRIMIDIN-3-YL]-PYRROLIDINE-1-CARBOXYLIC ACID [(S)-1-(4,4-DIMETHYL-CYCLOHEXYL)-PYRROLIDIN-3-YL]-METHYL-AMIDE

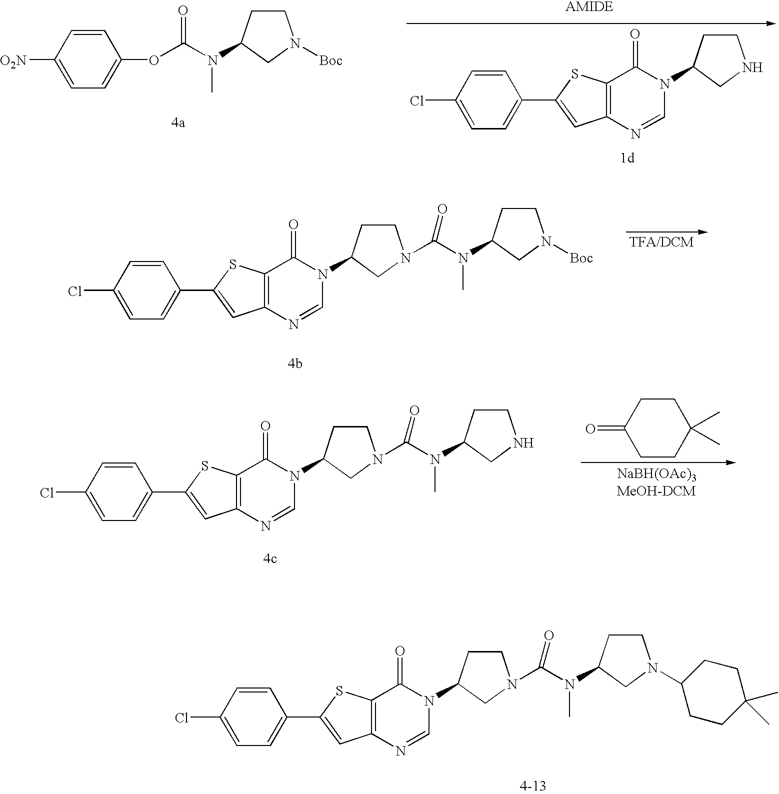 Step 4A:

To a pre-chilled solution of [(S)-1-Boc-pyrrolidin-3-yl]-methylamine (1.4 g, 7.0 mmol) in anhydrous THF (35 mL), was added TEA (2 mL) and 4-nitrophenyl chloroformate (1.7 g, 8.4 mmol) with stirring at room temperature for 30 minutes. The solvent was removed at reduced pressure, and the residue was dissolved in DCM (200 mL), washed well with NaHCO₃ (3×50 mL) and brine (2×50 mL), dried over MgSO₄, and concentrated at reduced pressure to afford ((S)-1-Boc-pyrrolidin-3-yl)-methyl-carbamic acid 4-nitrophenyl ester compound 4a (2.7 g).

Carbamic acid ester 4a (2.0 g, 5.6 mmol) and TEA (3.9 mL, 28 mmol) were added to a solution of thieno[3,2-d]pyrimidin-4-one compound 1d (1.87 g, 5.6 mmol) in DMA (30 mL). The reaction mixture was heated to 80° C. overnight. The solvent was removed at reduced pressure, and the residue was dissolved in DCM-isoPrOH (3:1, 300 mL), washed well with 0.5 N NaOH (5×50 mL), 10% NaHSO₄ (2×50 mL) and brine (2×50 mL), dried over MgSO4 and concentrated at reduced pressure to give a solid which was triturated with ether to afford compound 4b (3.1 g). LC/MS: 558 [MH]^{+.}

Step 4B:

TFA (10 mL) was added to an ice cooled solution of compound 4b (3.0 g, 5.34 mmol) in DCM (20 mL). Themixture was stirred for 1 hour. After removal of excess TFA by co-evaporation with DCM, the resulting oil was dissolved in DCM-isoPrOH (3:1, 240 mL) and treated with saturated NaHCO₃ solution (60 mL). The organic layer was separated and the water layer was extracted with DCM-isoPrOH (3:1, 80 mL). The combined organic layer was dried over MgSO₄ and evaporated to yield the unprotected compound 4c (1.85 g).

Step 4C:

To compound 4c (40 mg, 0.087 mmol) dissolved in MeOH-DCM (1 ML) was added a catalytic amount of HOAc, two equivalents of NaBH(OAc)₃, and 2 equivalents 4,4-dimethylcyclohexanone. The reaction mixture stirred for 1 hr at room temperature prior to quenching with H₂O (0.1 mL). The reaction mixture was filtered and purified by preparative LC/MS to give compound 4-13. LC/MS: 567.9 [MH]⁺.

By varying the alkylation reagent employed in Step 4C, the following compounds were prepared:

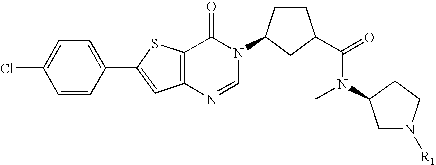

| | | | | |
|---|---|---|---|---|
| Cpd. | R₁ | MW | MS | RT* |

| | | | | |
|---|---|---|---|---|
| 4-1 | CH3 | 472.0 | 472.1 | 4.985 |
| 4-2 | 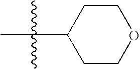 | 542.1 | 542.2 | 5.121 |
| 4-3 | 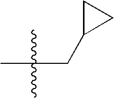 | 512.1 | 512.2 | 5.387 |
| 4-4 | 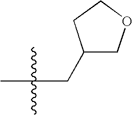 | 542.1 | 542.1 | 5.165 |
| 4-5 | 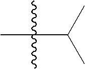 | 500.1 | 500.1 | 5.225 |
| 4-6 | 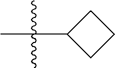 | 512.1 | 512.2 | 5.328 |
| 4-7 | 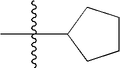 | 526.1 | 526.2 | 5.527 |
| 4-8 | 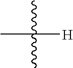 | 458.0 | 458.1 | 4.962 |
| 4-9 | 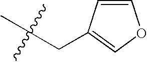 | 538.1 | 538.1 | 5.574 |
| 4-10 | 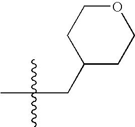 | 556.1 | 556.2 | 5.264 |
| 4-11 | 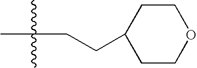 | 570.2 | 570.2 | 5.367 |
| 4-12 | 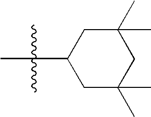 | 596.2 | 596.2 | 7.042 |
| 4-13 | 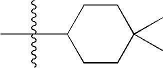 | 568.2 | 567.9 | 5.609 |

*All retention times (RT) reported for Analytical Method 1.

### EXAMPLE 5

### (R)-3-({(R)-3-[6-(4-CHLORO-PHENYL)-4-OXO-4H-THIENO[3,2-D]PYRIMIDIN-3-YL]-PYRROLIDINE-1-CARBONYL}-METHYL-AMINO)-PYRROLIDINE-1-CARBOXYLIC ACID TERT-BUTYL ESTER

By employing the R-enantiomer of compound 4a afforded by the method of Step 4A using [(R)-1-Boc-pyrrolidin-3-yl]-methylamine as starting material and the R-enantiomer of compound 1d afforded by the method of Steps 1C and 1D using (3R)-(+)-Boc-3-aminopyrrolidine as starting material, and by following the method described in Steps 4A through 4C with different alkylating reagents in the final addition step, the compounds of the following table were synthesized:

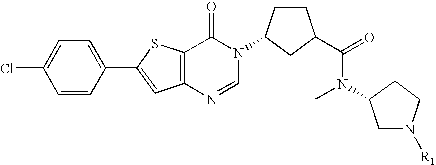

| Cpd. | R₁ | MW | MS | RT* |
|---|---|---|---|---|
| 5-1 | 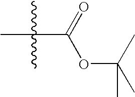 | 558.1 | 558.2 | 8.248 |
| 5-2 | 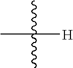 | 458.0 | 457.8 | 5.296 |
| 5-3 | 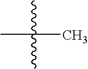 | 472.0 | 471.9 | 5.377 |
| 5-4 | 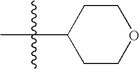 | 542.1 | 541.9 | 5.483 |
| 5-5 | 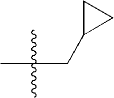 | 512.1 | 511.9 | 5.788 |
| 5-6 | 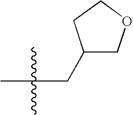 | 542.1 | 541.9 | 5.594 |
| 5-7 | 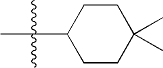 | 568.2 | 568.0 | 6.868 |
| 5-8 | 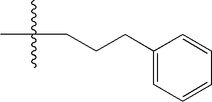 | 576.2 | 575.9 | 6.793 |
| 5-9 | 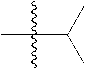 | 500.1 | 499.8 | 5.577 |

*All retention times (RT) reported for Analytical Method 1.

### EXAMPLE 6

### (R)-3-[6-(4-CHLORO-PHENYL)-4-OXO-4H-THIENO[3,2-D]PYRIMIDIN-3-YL]-PYRROLIDINE-1-CARBOXYLIC ACID ((S)-1-CYCLOPENTYL-PYRROLIDIN-3-YL)-METHYL-AMIDE

By employing compound 4a and the R-enantiomer of compound 1d afforded by the method of Steps 1C and 1D using (3R)-(+)-Boc-3-aminopyrrolidine as starting material, and by following the method described in Steps 4A through 4C with different alkylating reagents in the final addition step, the compounds of the following table were synthesized:

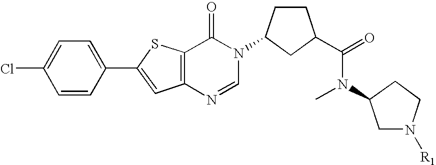

| Cpd. | R₁ | MW | MS | RT* |
|---|---|---|---|---|
| 6-1 | 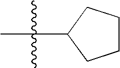 | 526.1 | 525.7 | 1.735 |
| 6-2 | 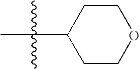 | 542.1 | 541.7 | 1.862 |
| 6-3 | 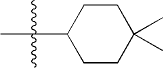 | 568.2 | 567.7 | 1.729 |
| 6-4 | 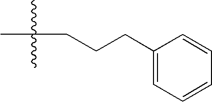 | 576.2 | 575.7 | 1.788 |
| 6-5 | 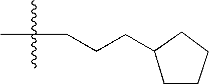 | 568.2 | 567.7 | 1.762 |

*All retention times RT) rfeported for Analytical Method 2.

### EXAMPLE 7

### (R)-3-({(S)-3-[6-(4-FLUORO-PHENYL)-4-oxo-4H-THIENO[3,2-D]PYRIMIDIN-3-YL]-PYRROLIDINE-1-CARBONYL}-METHYL-AMINO)-PYRROLIDINE-1-CARBOXYLIC ACID TERT-BUTYL ESTER

By employing the R-enantiomer of compound 4a afforded by the method of Step 4A using [(R)-1-Boc-pyrrolidin-3-yl]-methylamine as starting material and the 4-fluoro derivative of compound 1d afforded by the method of Steps 1C and 1D using methyl-3-amino-5-(4-fluorophenyl)-2-thiophene carboxylate as starting material, and by following the method described in Steps 4A through 4C with different alkylating reagents in the final addition step, the compounds of the following table were synthesized:

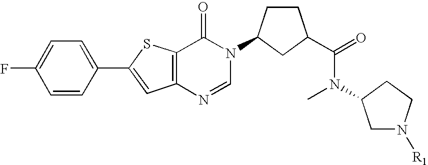

| Cpd. | R₁ | MW | MS | RT* |
|---|---|---|---|---|
| 7-1 | 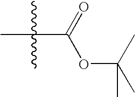 | 541.6 | 541.9 | 7.680 |
| 7-2 | 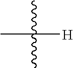 | 441.5 | 441.9 | 4.891 |
| 7-3 | 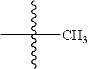 | 455.6 | 455.9 | 4.887 |
| 7-4 | 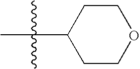 | 525.6 | 525.9 | 5.014 |
| 7-5 | 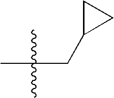 | 495.6 | 495.9 | 5.298 |
| 7-6 | 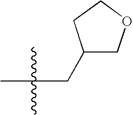 | 525.6 | 525.9 | 5.069 |
| 7-7 | 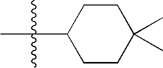 | 551.7 | 552.0 | 6.356 |
| 7-8 | 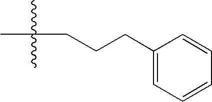 | 559.7 | 560.0 | 6.351 |
| 7-9 | 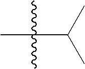 | 483.6 | 483.9 | 5.123 |

*All retention times (RT) reported for Analytical Method 1.

### EXAMPLE 8

### (S)-3-[6-(4-CHLORO-PHENYL)-4-oxo-4H-THIENO[3,2-D]PYRIMIDIN-3-YL]-PYRROLIDINE-1-CARBOXYLIC ACID [(R)-1-(3,3-DIMETHYL-2-OXO-BUTYL)-PYRROLIDIN-3-YL]-METHYL-AMIDE

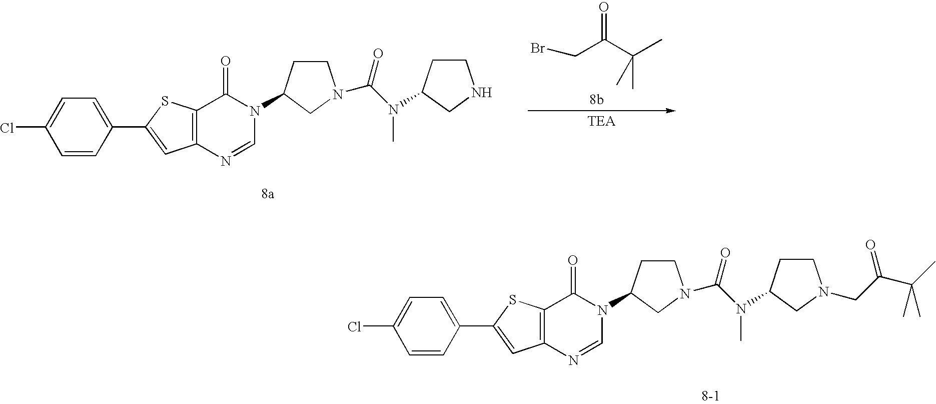 Step 8A:

Compound 8a, (S)-3-[6-(4-Chloro-phenyl)-4-oxo-4H-thieno[3,2-d]pyrimidin-3-yl]-pyrrolidine-1-carboxylic acid methyl-(R)-pyrrolidin-3-yl-amide, was synthesized and purified by the method of Steps 4A and 4B employing the R-enantiomer of the methylamide and compound 1d as reagents.

Step 8B:

To compound 8a (40 mg, 0.09 mmol) in MeOH (1 mL) was added compound 8b (2eq), TEA (2eq), and the reaction mixture was shaken at room temperature overnight. The reaction mixture was filtered and purified by preparative LC/MS to yield compound 8-1.

By varying the substitution and chirality of the reagents forming thiophene 8a as exemplified in Examples 5-7 and the alkylation reagent employed in Step 8B, the following compounds were prepared:

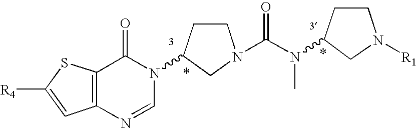

| | | | Stereo | | | |
|---|---|---|---|---|---|---|
| | | | Isomer | | | |
| Cpd. | R₄ | R₁ | (3, 3′) | MW | MS | RT* |
| 8-1 | 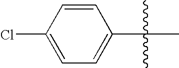 | 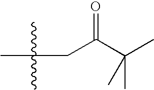 | (R, R) | 556.1 | 556.4 | 6.358 |
| 8-2 | 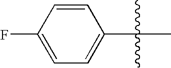 | 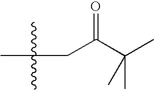 | (S, R) | 539.7 | 540.4 | 5.821 |
| 8-3 | 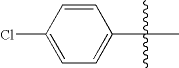 | 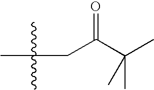 | (S, S) | 556.1 | 555.8 | 5.375 |
| 8-4 | 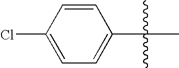 | 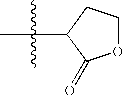 | (S, S) | 542.1 | 541.8 | 5.067 |
| 8-5 | 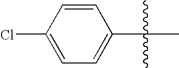 | 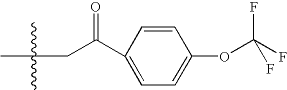 | (S, S) | 660.1 | 659.8 | 5.824 |

*All retention times (RT) reported for Analytical Method 1.

### EXAMPLE 9

### (S)-3-(4-OXO-6-P-TOLYL-4H-THIENO[3,2-D]PYRIMIDIN-3-YL)-PYRROLIDINE-1-CARBOXYLIC ACID METHYL-((S)-1-METHYL-PYRROLIDIN-3-YL)-AMIDE

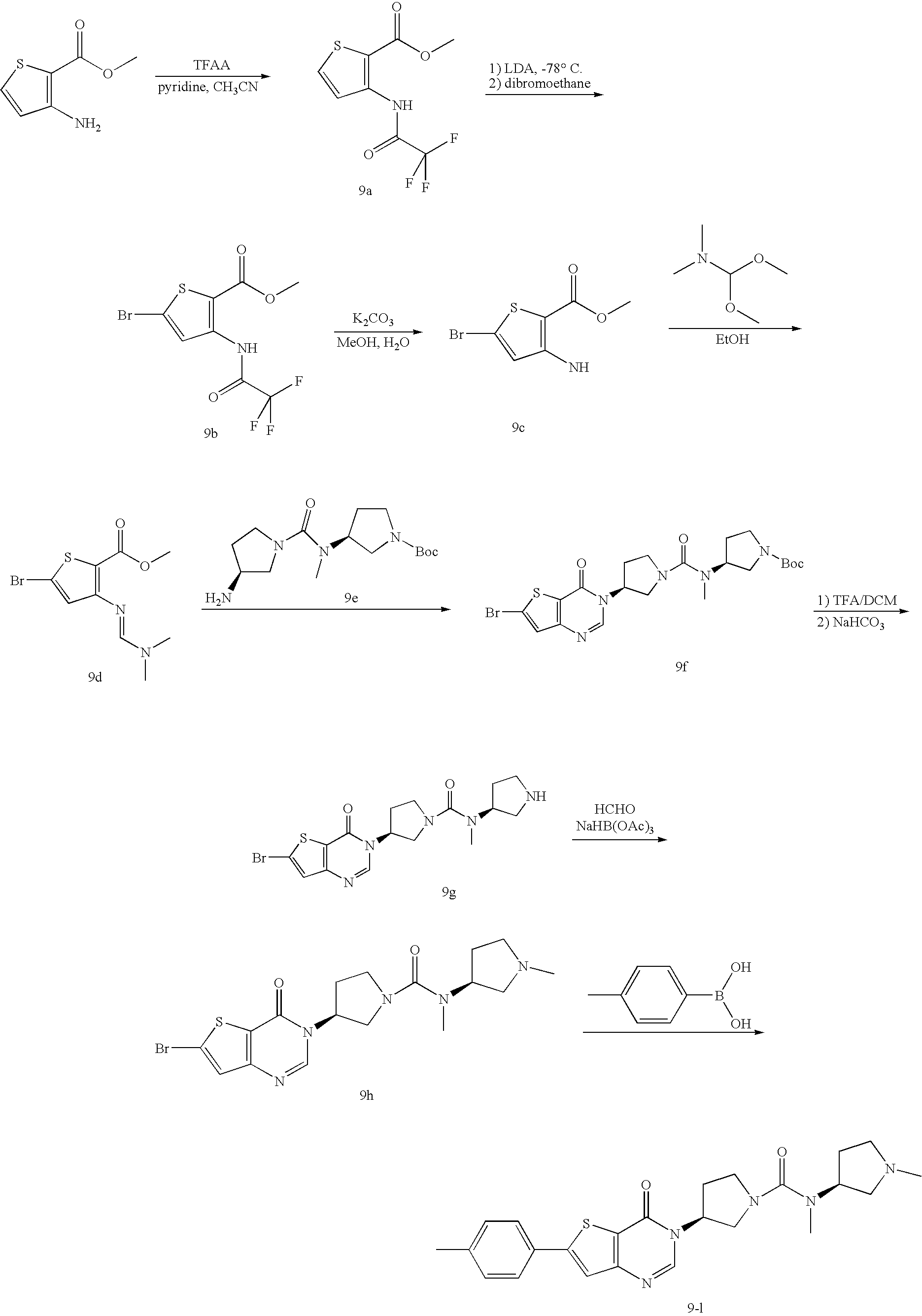 Step 9A:

Methyl 3-aminothiophene-2-carboxylate (10.0 g) in acetonitrile (130 mL) was cooled to 0° C. and treated with pyridine (6.2 mL) and trifluoroacetic anhydride (11.7 mL). After stirring for 5 minutes the reaction mixture was warmed to room temperature and stirred an additional 20 minutes. The reaction was poured into of ice water (1.5 L) and stirred for 15 minutes. The precipitate was collected by filtration and azeotroped with ethanol (3×200 mL) to yield compound 9a (15.9 g).

Step 9B:

To THF (100 mL) at −78° C. was added diisopropylamine (10 mL) and butyllithium (26.4 mL; 2.5 M in hexanes). The reaction mixture was warmed to 0° C. and stirred for 10 minutes. The reaction mixture was cooled to −78° C. and 9a (5.06 g) in THF (20 mL) was transferred via cannula. The reaction was stirred at −78° C. for 1 hour, and then treated with 1,2-dibromoethane (10.3 mL) in one portion. The reaction mixture was stirred at −78° C. for 30 minutes, then at room temperature for 30 minutes. Saturated sodium bicarbonate solution was added and the aqueous layer was extracted with three times with ethyl acetate. The combined organic layers were washed with water and brine. The organic layer was dried over MgSO₄, concentrated under reduced pressure, and purified by silica gel chromatography (2.5% ethyl acetate in hexanes) to give compound 9b (2.88 g).

Step 9C:

To 9b (2.83 g) in methanol (45 mL) was added potassium carbonate (5.89 g) in water (18 mL). The reaction was stirred for 3 hours, after which the solvent was removed under reduced pressure. The solid was partitioned between ethyl acetate and water. The ethyl acetate layer was washed with water and brine, dried over MgSO₄, and concentrated under reduced pressure to give compound 9c (1.97 g).

Step 9D:

To compound 9c (4.23g, 17.1 mmol) in ethanol (80 mL) was added dimethylformamide dimethyl acetal (5.9 mL, 44.2 mmol). The reaction mixture was stirred in a pressure vessel at 90° C. oil bath for 3 hours. Removal of solvent and excess dimethylformamide dimethyl acetal by co-evaporated with toluene gave compound 9d (5.1g).

Step 9E:

To the solution of compound 4a (16.0 g, 43.8 mmol) in DMA (150 mL) was added (S)-pyrrolidin-3-ylamine (4.1 g, 48.2 mmol) and TEA (30.7 mL, 219 mmol). The reaction mixture was heated in a pressure vessel at 70° C. overnight. The solvent was removed at reduced pressure and the residue was dissolved in DCM-isoPrOH (3: 1, 800 mL), washed well with 1 N NaOH (5×100 mL), brine (2×100 mL) and dried over MgSO₄ then concentrated at reduced pressure. Crystallization with ether-hexanes afforded compound 9e (10.0 g), LC/MS: 313.2 [M+H]⁺.

To compound 9d resulting from the previous step in ethanol (80 mL) was added compound 9e (6.44 g, 21.2 mmol). The reaction mixture was stirred in a pressure vessel at 90° C. for 2 days, monitored by LC/MS. The reaction mixture was subjected to rotary evaporation to remove solvent. The resulting residual was dissolved in 800 mL DCM, washed with 10% NaHSO₄, saturated NaHCO₃ solution, and brine; dried over MgSO₄, filtered and concentrated under reduced pressure to give an orange oil. The crude product was purified by silica gel column chromatography (from 0% MeOH in DCM to 2% MeOH in DCM) to afford 9f (3.0g). LC/MS: 526.1 [MH]⁺.

Step 9F:

To compound 9f (3.0 g, 5.7 mmol) in DCM (50 mL), cooled in an ice bath was added TFA. The reaction mixture was stirred for 0.5 hr to effect removal of the Boc protecting group. After removal of excess TFA by co-evaporation with DCM, the resulting oil was dissolved in DCM-isoPrOH (3:1, 240 mL) and basified with saturated NaHCO₃ solution (100 mL). The organic layer was separated and the water layer was extracted with DCM-isoPrOH (3:1, 80 mL). The combined organic layer was dried over MgSO₄ and subjected to rotary evaporated to yield the unprotected compound 9g (1.67g), LC/MS: 426.0 [MH]⁺.

Step 9H:

To compound 9g (875 mg, 2.05 mmol) dissolved in MeOH-DCM (10 mL) was added a catalytic amount of HOAc, 3 equivalents formaldehyde (37% in H₂O) and two equivalents of NaBH(OAc)₃. After 1 h at room temperature, the reaction mixture was subjected to rotary evaporation. The resulting oil was dissolved in DCM-isoPrOH (3:1, 120 mL) and washed with saturated NaHCO₃ solution (50 mL). The organic layer was separated and the water layer was extracted with DCM-isoPrOH (3:1, 50 mL). The combined organic layer was washed with brine, dried over MgSO₄, filtered, and subjected to rotary evaporation to yield compound 9h (0.88g), LC/MS: 440.0 [MH]⁺.

Step 9I:

To compound 9h (410 mg, 0.93 mmol) in 10 mL 1,4-dioxane was added 4-methylphenylboronic acid (190 mg, 1.4 mmol), Na₂CO₃ (245 mg, 2.3 mmol) in H₂O (5 mL). The mixture was flushed with N₂ for 5 min, added Pd(PPh₃)₄ (86 mg, 0.07 mmol) and flushed for another 5 min and was then stirred at 110° C. for 5h. The reaction mixture was filtered and rinsed with 1,4-dioxane. The filtrate was diluted with DCM-isoPrOH (3:1, 120 mL) and washed with saturated NaHCO₃ solution (30 mL). The organic layer was separated and the water layer was extracted with DCM-isoPrOH (3:1, 60 mL). The combined organic layer was washed with brine, dried over MgSO₄, filtered, and subjected to rotary evaporation to yield oil (0.48 g). The crude product was purified by silica gel column chromatography (from 3% to 5% of 2N NH₃ in MeOH over DCM) to afford compound 9-1 (0.36g). LC/MS: 452.3 [MH]⁺.

By varying the substitutions of the boronic acid in Step 91 and the alkylation reagent employed in Step 9H, the following compounds were prepared:

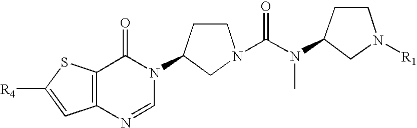

| | | | | | | HPLC |
|---|---|---|---|---|---|---|
| Cpd. | R₄ | R₁ | MW | MS | RT | Method |
| 9-1 | 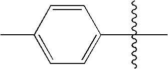 | 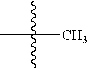 | 451.6 | 452.3 | 4.704 | 1 |
| 9-2 | 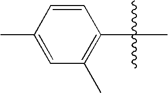 | 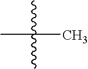 | 465.6 | 466.4 | 4.769 | 1 |
| 9-3 | 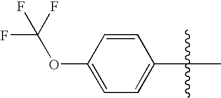 | 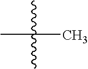 | 521.6 | 522.4 | 4.999 | 1 |
| 9-4 | 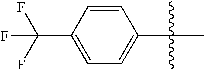 | 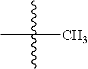 | 505.6 | 506.3 | 5.024 | 1 |
| 9-5 | 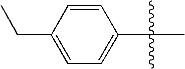 | 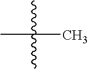 | 465.6 | 466.3 | 4.978 | 1 |
| 9-6 | 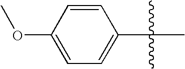 | 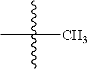 | 467.6 | 468.4 | 4.572 | 1 |
| 9-7 | 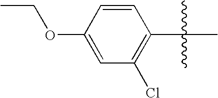 | 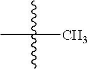 | 516.1 | 516.3 | 5.105 | 1 |
| 9-8 | 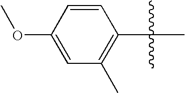 | 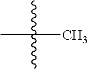 | 481.6 | 482.4 | 4.803 | 1 |
| 9-9 | 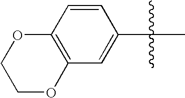 | 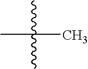 | 495.6 | 496.4 | 4.785 | 1 |
| 9-10 | 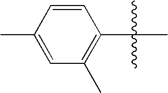 | 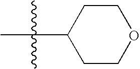 | 535.7 | 536 | 4.652 | 1 |
| 9-11 | 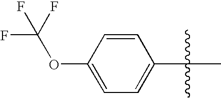 | 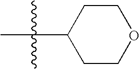 | 591.7 | 592 | 4.728 | 1 |
| 9-12 | 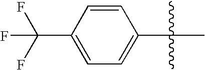 | 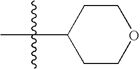 | 575.7 | 576 | 4.682 | 1 |
| 9-13 | 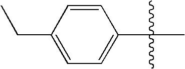 | 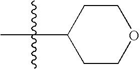 | 535.7 | 536 | 4.833 | 1 |
| 9-14 | 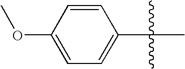 | 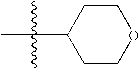 | 521.7 | 522 | 4.544 | 1 |
| 9-15 | 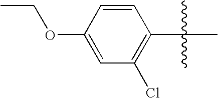 | 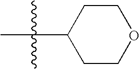 | 586.2 | 586 | 1.792 | 2 |
| 9-16 | 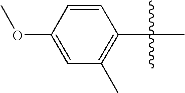 | 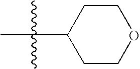 | 551.7 | 552 | 4.482 | 1 |
| 9-17 | 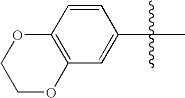 | 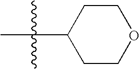 | 565.7 | 566 | 1.851 | 2 |

### EXAMPLE 10

### (S)-3-[6-(4-CHLORO-PHENYL)-4-OXO-4H-THIENO[3,2-D]PYRIMIDIN-3-YL]-PYRROLIDINE-1-CARBOXYLIC ACID METHYL-[(S)-1-(3-PHENYL-PROPYL)-PYRROLIDIN-3-YL]-AMIDE

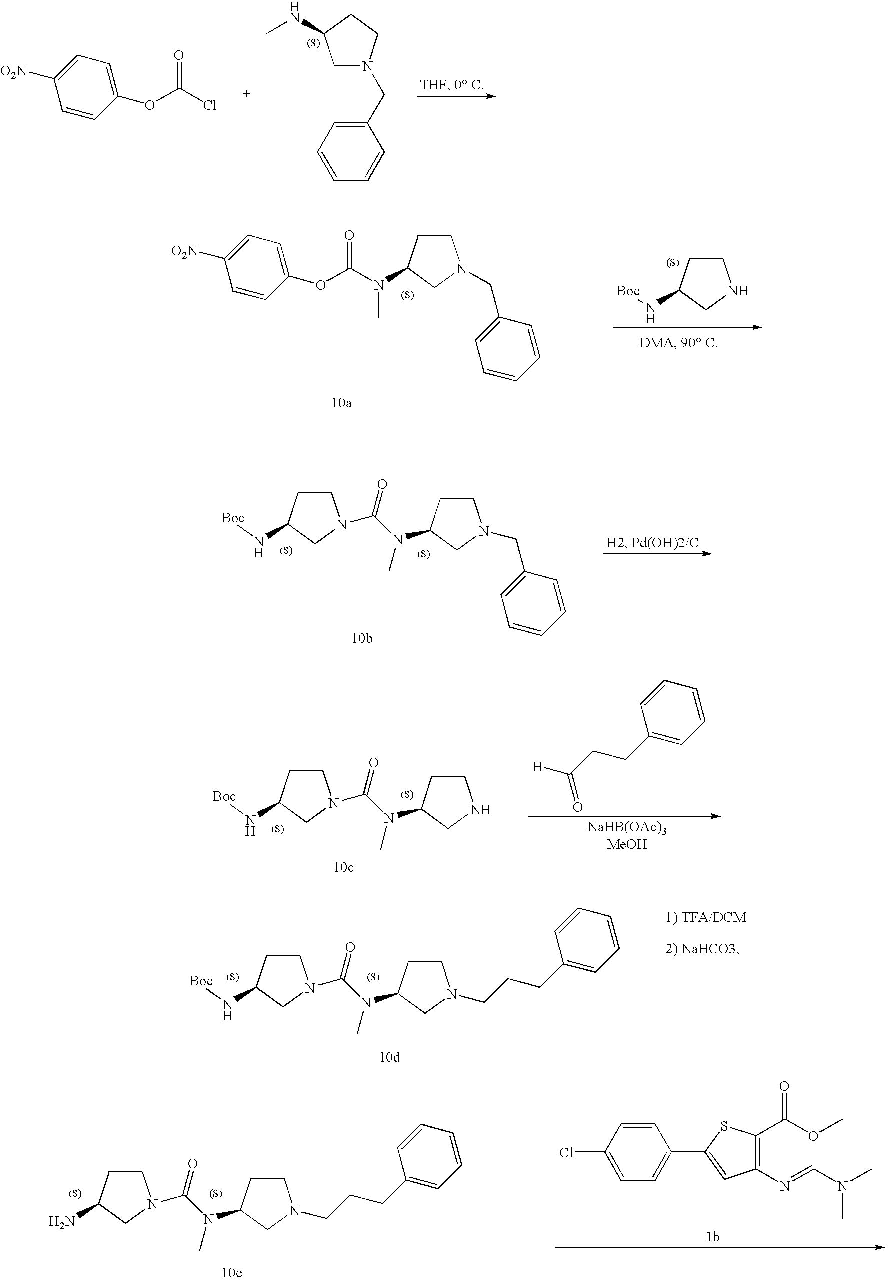 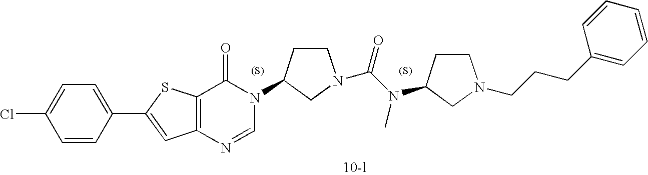 Step 10A:

4-Nitrophenyl chlorocarbonate (10.85 g, 54.0 mmol) was dissolved in THF (90 mL) and chilled to 0° C. 3S-(methylamino)-1-benzyl-pyrrolidine (8.5 g, 44.7 mmol) was dissolved in dry THF (20 mL) and added dropwise. The reaction was stirred at room temperature for 30 minutes. The reaction was filtered, washed with a small amount of THF and dried to give the hydrochloride salt 10a (15.58 g).

Step 10B:

A mixture of 10a (15.37 g, 39.2 mmol) and (3S)-(−)-3-(tert-butoxycarbonylamino)pyrrolidine (7.3 g, 39.2 mmol) was suspended in DMF (100 mL) and treated with DIEA (13.6 mL, 78.5 mmol). The mixture was placed in a 100° C. oil bath and heated under nitrogen atmosphere for 5 hours. DCM/isoPrOH (3:1) was added and the solution was washed well with 0.5 N NaOH solution followed by brine, dried over MgSO₄ then concentrated to a syrup. Crystallization from EtOAC provided compound 10b (9.5 g) as an off-white solid. LC/MS: 403 [MH]⁺.

Step 10C:

Compound 10b (8.0 g, 20 mmol) was dissolved in methanol (150 mL), degassed with nitrogen, treated with palladium hydroxide 20% on carbon (60% wet, 1.6 g) and hydrogenated at 40 psi at room temperature. After shaking for approximately 5 hours, the solution was filtered through Celite® and washed with methanol. Evaporation of the solvent and titituration with acetonitrile provided compound 10c (6.5 g). LC/MS: 313 [MH]⁺.

Step 10D:

Compound 10c (6.2 g, 20 mmol) was dissolved in methanol (100 mL) and treated with acetic acid (0.3 mL). Sodium triacetoxyborohydride (8.5 g, 40 mmol) was added followed by the dropwise addition of 3-phenylpropionaldehyde (5.48 g, 40 mmol) in methanol (40 mL). After 2 hours, a small amount of water was added. The reaction was diluted with EtOAc (800 ml), washed with saturated sodium bicarbonate solution and saturated sodium chloride and dried over MgSO₄. After evaporation of solution in vacuo, the material was suspended in ether and treated carefully with 1 equivalent HCl to provide 10d (8.9 g) as the hydrochloride salt. LC/MS: 431 [MH]⁺.

Step 10E:

Compound 10d (8.6 g) was dissolved in 50 mL DCM and chilled in an ice bath, and to this solution was added trifluoroacetic acid (50 mL) dropwise. After stirring for 30 minutes, the solvent was evaporated and compound 10e was obtained as the TFA salt, which was dissolved in EtOAc and washed with 1 N NaOH solution. Back extraction of the EtOAc and combination and drying of organic layers provided the free base of 10e (6.6 g) as an oil with some entrained solvent. LC/MS: 330 [MH]⁺. This material was used without further purification.

Step 10F:

Reaction of compounds 10e and 1b employing the method of Step 1B afforded after purification compound 10-1.

By varying the substitutions of the starting thiophene 1a, the following compounds were prepared:

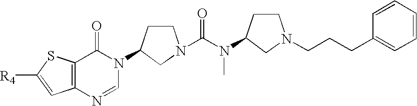

| | | | | | HPLC |
|---|---|---|---|---|---|
| Cpd. | R₄ | MW | MS | RT | Method |
| 10-1 | 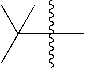 | 521.7 | 522 | 1.669 | 2 |
| 10-2 | 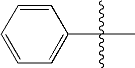 | 541.7 | 542 | 5.844 | 1 |
| 10-3 | 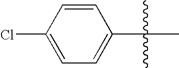 | 576.2 | 576 | 1.519 | 2 |
| 10-4 | 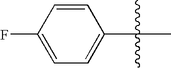 | 559.7 | 560 | 1.600 | 2 |
| 10-5 | 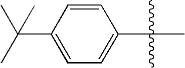 | 597.8 | 598 | 7.207 | 1 |

### EXAMPLE 11

### (S)-3-[4-OXO-6-(4-TRIFLUOROMETHYL-PHENYL)-4H-THIENO[3,2-D]PYRIMIDIN-3-YL]-PYRROLIDINE-1-CARBOXYLIC ACID METHYL-[(S)-1-(3-PHENYL-PROPYL)-PYRROLIDIN-3-YL]-AMIDE

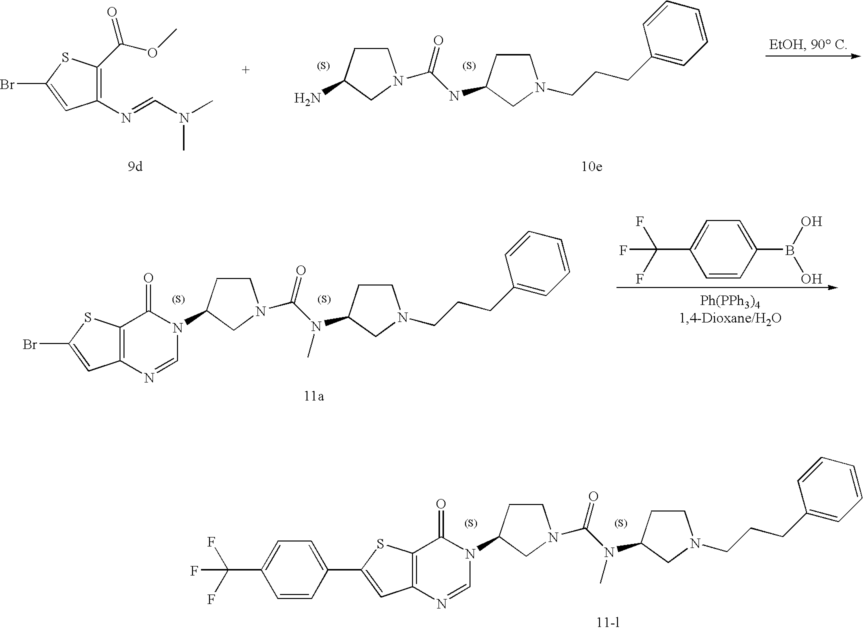 Step 11A:

Compound 11a, (S)-3-(6-bromo-4-oxo-4H-thieno[3,2-d]pyrimidin-3-yl)-pyrrolidine-1-carboxylic acid methyl-[(S)-1-(3-phenyl-propyl)-pyrrolidin-3-yl]-amide, was synthesized by the method of Step 9E employing reagents 9d and 10e.

Step 11B:

Compound 11a was subjected to Suzuki coupling conditions of the method of Step 9I employing 4-(trifluoromethyl)phenylboronic acid to afford after purification compound 11-1.

By varying the substitutions of the phenylboronic acid in Step 11B, the following compounds were prepared:

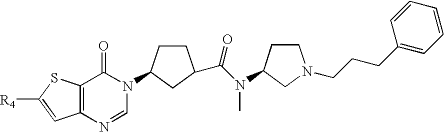

| Cpd. | R₄ | MW | MS | RT* |
|---|---|---|---|---|
| 11-1 | 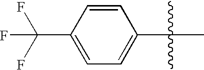 | 609.7 | 610 | 4.988 |
| 11-2 | 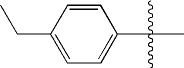 | 569.8 | 570 | 5.494 |
| 11-3 | 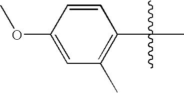 | 585.8 | 586 | 5.113 |

*All retention times (RT) reported for Analytical Method 1.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having the following structure: 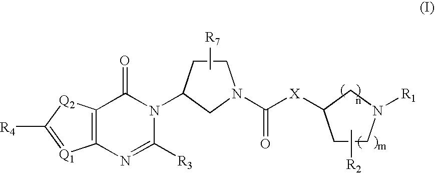
or a pharmaceutically acceptable salt, ester, solvate, stereoisomer, or prodrug thereof,
wherein:
m is 0 or 1;
n is 1 or 2;
Q₁ is ═CH— or ═N—;
Q₂ is —S—, —O—, —C(R₅)═C(R₆)—, —C(R₅)═N—, or —N═C(R₆)—;
R₁ is alkyl, substituted alkyl, aryl, substituted aryl, heteroaryl, substituted heteroaryl, heteroarylalkyl, substituted heteroarylalkyl, arylalkyl, substituted arylalkyl, heterocyclealkyl, or substituted heterocyclealkyl;
R₃ is hydrogen, halogen, cyano, alkyl, substituted alkyl, arylalkyl, or substituted arylalkyl;
R₄ is hydrogen, alkyl, substituted alkyl, aryl, substituted aryl, arylalkyl, substituted arylalkyl, heterocycle, substituted heterocycle, heterocyclealkyl, or substituted heterocyclealkyl;
R₂, R₅, R₆ and R₇ are the same or different and independently hydrogen, alkyl, or substituted alkyl;
X is —CH₂— or —N(R₈)—; and
R₈ is hydrogen, alkyl, or substituted alkyl.

2. A compound according to claim 1 wherein X is —CH₂—.

3. A compound according to claim 1 wherein X is —N(R₈)—.

4. A compound according to claim 1 wherein n is 1.

5. A compound according to claim 1 where n is 2.

6. A compound according to claim 4 wherein m is 0.

7. A compound according to claim 4 wherein m is 1.

8. A compound according to claim 5 wherein m is 1.

9. A compound according to claim 1 wherein Q₁ is ═CH—.

10. A compound according to claim 1 wherein Q₁ is ═N—.

11. A compound according to claim 1 wherein Q₂ is —S—.

12. A compound according to claim 1 wherein Q₂ is —O—.

13. A compound according to claim 1 wherein Q₂ is —C(R₅)═C(R₆)—.

14. A compound according to claim 1 wherein Q₂ is —C(R₅)═N—.

15. A compound according to claim 1 wherein Q₂ is —N═C(R₆)—.

16. A compound according to claim 1 wherein R₄ is substituted aryl.

17. A compound according to claim 16 wherein R₄ is substituted phenyl.

18. A compound according to claim 17 wherein said substituted phenyl is phenyl substituted with fluoro, chloro, methyl, —OCF₃, or CF₃.

19. A compound according to claim 18 wherein said substituted phenyl is 4-fluorophenyl, 4-chlorophenyl, 4-methylphenyl, 4-trifluoromethoxyphenyl or 4-trifluoromethylphenyl.

20. A compound according to claim 19 wherein said substituted phenyl is 4-fluorophenyl or 4-chlorophenyl.

21. A pharmaceutical composition comprising a compound according to claim 1 and a pharmaceutically acceptable carrier or diluent.

22. A method for inhibiting the activity of melanin concentrating hormone receptor in a mammal, comprising administering to the mammal a pharmaceutical composition according to claim 21.

23. A method for treating obesity in a mammal, comprising administering to the mammal a pharmaceutical composition according to claim 21.

24. A method for treating anxiety in a mammal, comprising administering to the mammal a pharmaceutical composition according to claim 21.

25. A method for treating depression in a mammal, comprising administering to the mammal a pharmaceutical composition according to claim 21.
